(19) United States
(12) Patent Application Publication
(10) Pub. No.: US 2025/0249152 A1
(43) Pub. Date: Aug. 7, 2025

(54) METHODS AND COMPOSITIONS FOR GENERATING BONE AND CARTILAGE

(71) Applicant: Hofstra University, Hempstead, NY (US)

(72) Inventor: Roche Rey C. deGuzman, Copiague, NY (US)

(73) Assignee: Hofstra University, Hempstead, NY (US)

(21) Appl. No.: 19/044,232

(22) Filed: Feb. 3, 2025

Related U.S. Application Data
(60) Provisional application No. 63/549,223, filed on Feb. 2, 2024.

(51) Int. Cl. A61L 27/54

(52) U.S. Cl. CPC A61L 27/54; A61L 2300/252; A61L 2430/02; A61L 2430/06

(57) ABSTRACT

The present invention provides methods of generating bone and cartilage in vivo. The methods include delivering leukocyte cell-derived chemotaxin-1 (LECT-1) biomolecules and bone morphogenetic protein-2 (BMP-2) biomolecules into an in vivo target site, preferably by using biocompatible scaffolds containing the biomolecules.

This application claims the benefit of the filing date of U.S. Provisional application 63/549,223, filed on Feb. 2, 2024, the contents of which is incorporated herein by reference in its entirety.

### BACKGROUND OF THE PRESENT INVENTION

When bone tissue sustains damage, such as a fracture, mesenchymal stem cells and progenitor cells in the periosteum and surrounding tissues get activated, migrate, proliferate and differentiate into osteoblasts (bone progenitor cells) to ultimately regenerate bone. In most cases, stem cells differentiate into chondroblasts (cartilage progenitor cells) and cartilage prior to the formation of osteoblasts and bone. For minor damage, immobilization of the bone at the affected area allows stem cells and osteoblasts activation, thereby the immediate area is repaired. In circumstances where stem cells, chondroblasts, and osteoblasts cannot be activated effectively, such as in the case of a complex fracture, critical-size defect (bone gap is too large), damage in a joint or damage in combination with osteomyelitis and surgical removal of damaged tissues, autologous bone grafting has been used as a standard method for repairing damage or deficits. When the defective region is too large to repair with autologous bone, osteoinductive and osteoconductive materials may be used in combination with autologous bone. However, in humans, sources of autologous bone are limited. In addition, supplying autologous bone is accompanied by high costs and pain to the donor. Moreover, the use of autologous bone causes a new deficit in a region (donor site morbidity) which was originally normal and from which the autologous bone is obtained. Also, a further procedure is required to collect bone, wherein the amount of the bone which can be collected is limited. Clearly, more effective methods are needed to regenerate cartilage and bones, especially in cases of complex fractures, nonunions, extensive bone damage, or damage in joints, or damage in combination with diseases requiring surgical removal, and particularly where autologous bone transplantation is not possible.

### SUMMARY OF THE PRESENT INVENTION

In one embodiment, the present invention provides a method of generating bone in vivo. The method comprises administering a leukocyte cell-derived chemotaxin-1 (LECT-1) biomolecule and a bone morphogenetic protein-2 (BMP-2) biomolecule into an in vivo target site, whereby bone is generated in the in vivo target site.

In one embodiment, the LECT-1 and BMP-2 biomolecules are administered by placing the biomolecules into a biocompatible scaffold. The scaffold can comprise a natural polymer, synthetic polymer, ceramic biomaterial, porous metal biomaterial and combinations thereof. The natural polymer can be a protein-based based polymer, polysaccharide-based polymer and combinations thereof. The protein-based based polymer can be collagen, gelatin, fibrin, fibronectin, elastin, silk fibroin, hair protein extract, residual hair, laminin, polypeptides and combinations thereof. The polysaccharide-based polymer can be a chitosan, cellulose, cellulose acetate, dextran, alginic acid/alginate, non-sulfated glycosaminoglycan (GAG), sulfated GAG, proteoglycans, proteoglycan complex, extracellular matrix (ECM) extracts and/or decellularized tissue ECM material and combinations thereof. An example of a non-sulfated GAG is hyaluronic acid. Examples of sulfated GAG are chondroitin sulfate, dermatan sulfate, keratan sulfate, heparan sulfate and heparin. Examples of proteoglycans are decorin, biglycan, testican, bikunin, fibromodulin, lumican, aggrecan, perlecan, betaglycan, agrin, neurocan, versican and brevican. Examples of a proteoglycan complex are heparan sulfate proteoglycan, chondroitin sulfate proteoglycan and keratan sulfate proteoglycan. Examples of synthetic polymers are polyethylene (PE), polypropylene (PP), polytetrafluoroethylene (PTFE), polyvinyl chloride (PVC), polydimethylsiloxane (PDMS), polymethyl methacrylate (PMMA), polyhydroxyethyl methacrylate (PHEMA), polyethylene terephthalate (PET), nylon, polystyrene (PS), polyether ether ketone (PEEK), polyacrylic acid (PAA), polylactic acid (PLA), polyglycolic acid (PGA), poly(lactic-co-glycolic acid) (PLGA), poly(caprolactone) (PCL), polypropylene fumarate (PPF), polydioxanone (PDO), polyorthoester (POE), polyanhydride (PAH), polyurethane (PUR), polyamidoamine (PAA), polyvinyl alcohol (PVA), polyvinyl acetate (PVAc), poly(ethylene glycol) (PEG), modified PEG, polyacrylonitrile (PAN), polycyanoacrylate (PCA), poly(ethylene-vinyl acetate) (PEVA), polyacrylate, cyanoacrylate polymer, and combinations thereof. Examples of ceramic biomaterials are alumina, zirconia, metal oxides, carbon, carbides, glass−/− silica, silicon nitride, silicon carbide, porcelain, bioglass−/−bioactive glass, calcium phosphates, hydroxyapatite (HA), carbonated HA, beta-tricalcium phosphate (TCP), calcium carbonate, glass ceramic composites and combinations thereof. Examples of porous metal biomaterials are titanium, titanium alloys, nitinol (Ti—Ni)−/−shape memory alloy, tantalum, tantalum alloys, platinum, iron, zinc, magnesium, stainless steel, cobalt chromium alloys, and combinations thereof.

In one embodiment, the LECT-1 and BMP-2 biomolecules are incorporated into the scaffold by covalent and/or non-covalent interactions. In one embodiment, the covalent interaction creating covalent bonds between the scaffold and BMP-2 and/or between the scaffold and LECT-1 and/or within the scaffold itself is created by exposing the scaffold to a photo (light)-based reaction, thiol-ene reaction, disulfide oxidation reaction, crosslinking reaction using crosslinkers and combinations thereof. In one embodiment, the non-covalent interactions are created by exposing the scaffold to physical mixing, dissolution, entanglement, absorption, adsorption, ionic or charged or electrostatic interaction, hydrophobic interaction, 3D filament printing, 3D resin printing and combinations thereof.

In one embodiment, the target site is a bone fracture, bone defect and/or bone gap. In one embodiment, the target site is a skin layer, under the skin or within muscle tissue which provides a source for autograft bone tissue, wherein the skin layer is a subcutaneous layer, hypodermis, adipose tissue, subcutaneous or white adipose tissue (sWAT). In one embodiment, the bone to be regenerated is trabecular bone or cortical bone.

In one embodiment, the LECT-1 biomolecule is a LECT-1 protein and/or a nucleic acid molecule encoding a LECT-1 protein. The nucleic acid molecule encoding a LECT-1 protein is DNA or mRNA. In one embodiment, the BMP-2 biomolecule is a BMP-2 protein and/or a nucleic acid molecule encoding a BMP-2 protein. The nucleic acid molecule encoding a BMP-2 protein is DNA or mRNA.

In one embodiment, the present invention provides a method of generating cartilage in vivo. The method comprises providing a biocompatible scaffold containing leukocyte cell-derived chemotaxin-1 (LECT-1) biomolecule and placing the biocompatible scaffold into an in vivo target site, wherein cartilage is generated within the target site. In some embodiments, the biocompatible scaffold further contains bone morphogenetic protein-2 (BMP-2) biomolecule. Examples of cartilage to be regenerated include articular cartilage, endochondral cartilage, hyaline cartilage, elastic cartilage, fibrous cartilage/fibrocartilage and combinations thereof. Examples of the target site includes a cartilage defect, cartilage gap for cartilage regeneration. The target site can also be a skin layer, under the skin or within muscle tissue to provide a source for autograft cartilage tissue, wherein the skin layer is a subcutaneous layer, hypodermis, adipose tissue, subcutaneous or white adipose tissue (sWAT).

In one embodiment, the present invention provides a biocompatible scaffold containing leukocyte cell-derived chemotaxin-1 (LECT-1) biomolecule and bone morphogenetic protein-2 (BMP-2) biomolecule, or containing LECT-1, for generating bone and/or cartilage in vivo.

### BRIEF DESCRIPTION OF THE DRAWINGS

FIG. 1: Proof of Generating Ectopic Bone: CT Scan. A) Scaffolds, without (−) and with growth factors: LECT-1 and BMP-2 at 1-50 μg/kg body weight and 100-600 μg/kg, respectively, were intramuscularly implanted in mice. B) Representative computed tomography (CT)-reconstructed images showing new interconnected more compact bone tissues (ectopic ossification) at 2 weeks for scaffolds with LECT-1 and BMP-2. In comparison, no new bones were formed in negative control samples (scaffolds only).

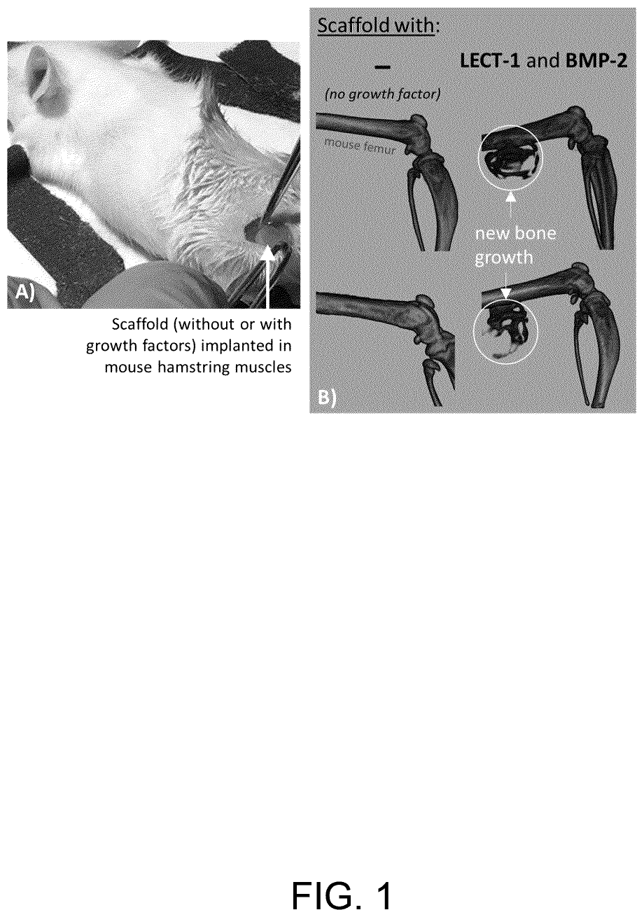

FIG. 2: Proof of Generating Ectopic Bone: Histology. Masson's trichrome (MT)-stained, processed, and decalcified representative scaffold with LECT-1 and BMP-2 growth factors, implanted in mice hamstring muscles and retrieved after 2 weeks. MT: blue=collagen and tissue extracellular matrix (ECM), red=muscle and cytosol of cells, dark purple=nuclei of cells. Collagen ECM is observed after decalcification of the new bone tissue growth. The wavy pattern and spaces filled with cells indicate trabecular bone with bone marrow-like contents. Embedded in the bone matrix are the osteocytes or bone cells with occasional blood vessels.

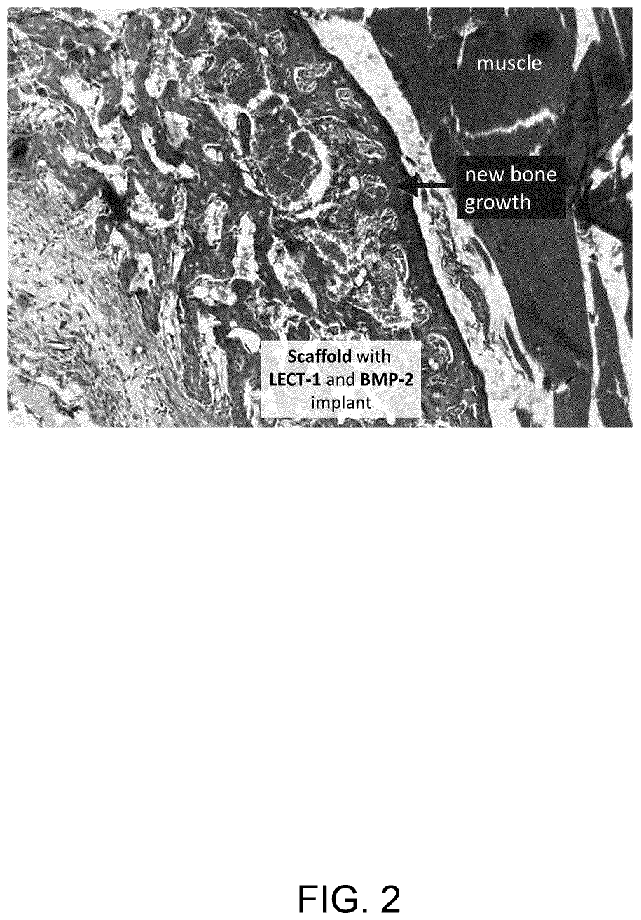

FIG. 3: Proof of Generating Ectopic Cartilage: Histology. Masson's trichrome (MT) (left) and toluidine blue (TB) (right)-stained representative scaffold with LECT-1 only growth factor, implanted in mice hamstring muscles and retrieved after 2 weeks. MT: blue=collagen and tissue ECM, red=muscle and cytosol of cells, dark purple=nuclei of cells. TB: blue=tissue ECM rich in proteoglycans and glycosaminoglycans (GAGs). Cells and tissue morphology and staining confirmed the induction of cartilage formation or chondrogenesis using LECT-1 delivered through a gel scaffold. Some chondroblast cells can be seen undergoing cell division or mitosis. No blood vessels were observed in the tissue sample. Cartilage tissue ECM is rich in proteoglycans and GAGs (right, blue signals) for increased water absorption, lubrication, and shock-absorption.

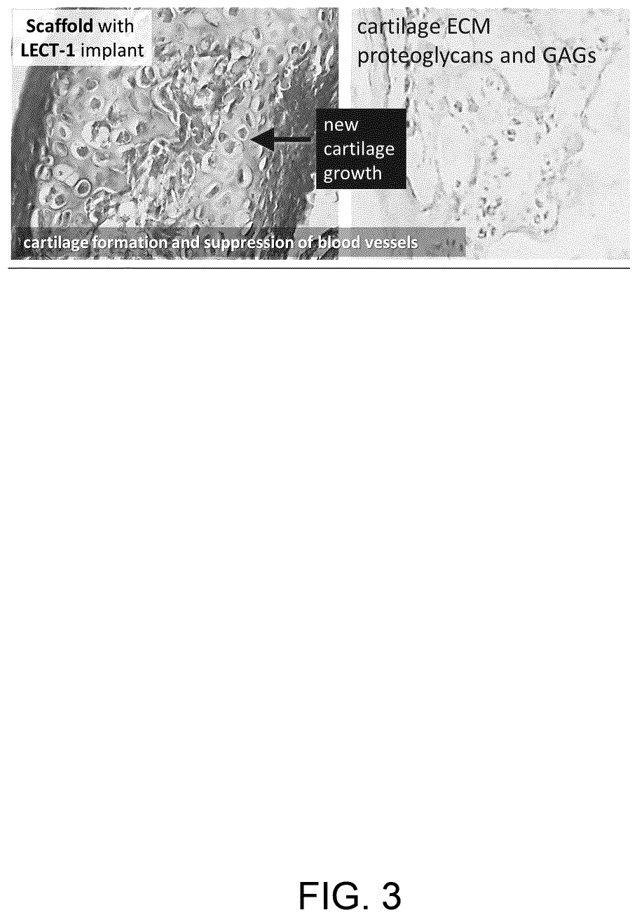

FIG. 4: Proof of Bone Regeneration of a Critical-Size Defect. Keratin-based scaffold (KTN), specifically as residual hair gel strands at approximately 50 μL are absorbed with 200 ng of LECT-1 and 5 μg of BMP-2 growth factors, then used as a patch to a critical-size calvarial (skull) circular defect with a diameter of 4 mm of a parietal bone above the dura mater. After closing the skin and 8 weeks of mouse recovery, reconstructed CT scans show that the empty group (defect left untreated) and the KTN only group (without growth factors) did not heal the bone gap while the KTN with LECT-1 and BMP-2 group has closed or regenerated the bone defect in 4/4 biological replicates (two shown above).

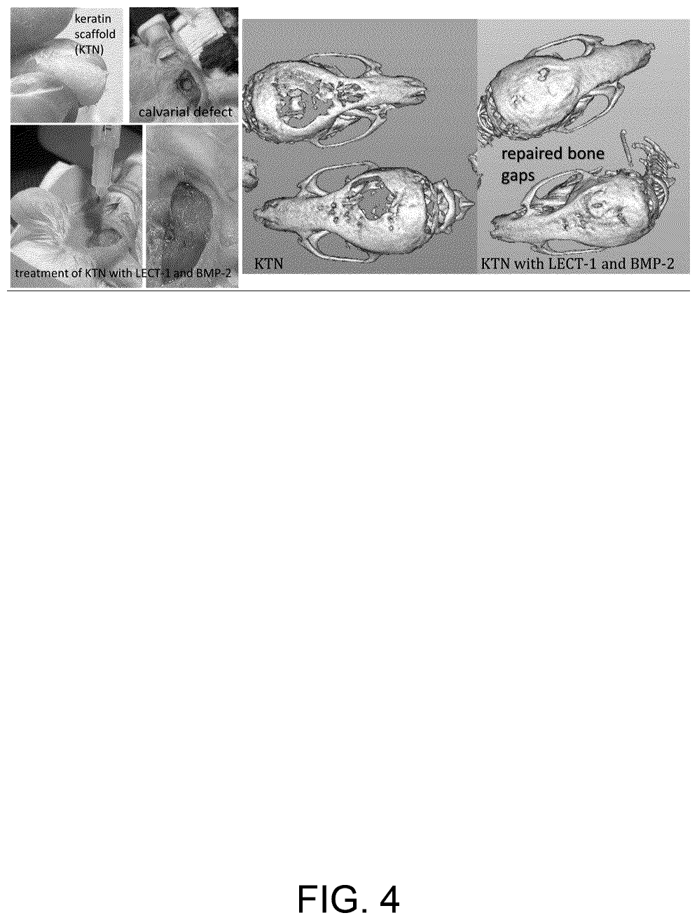

FIG. 5: Proof of Generating Custom-Shaped Ectopic Cartilage: 3D-Printing and Macroscopic Visualization. 3D-printing using photo-polymerizable resin mixed with BMP-2 and LECT-1 growth factors can be fabricated and shaped into a human ear and a curved disk with holes (simulation of articular cartilage). After 2 months inside the subcutaneous tissue (under the dorsal skin) of mice, the scaffold can now be seen with deposited cartilage tissues.

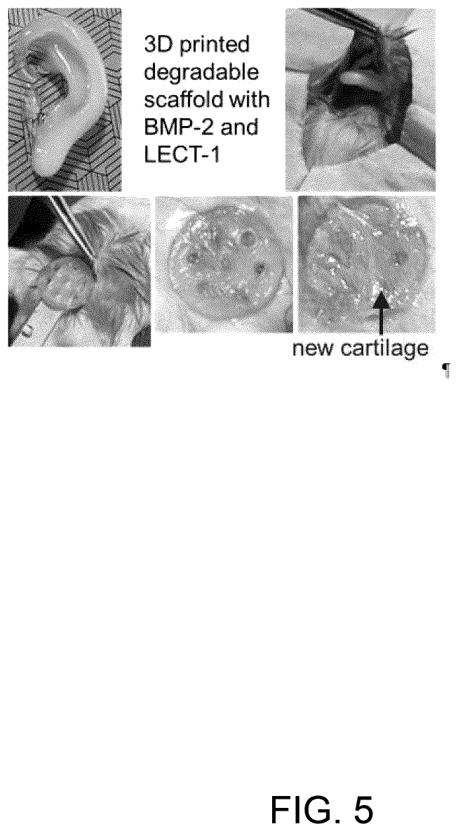

### DETAILED DESCRIPTION OF THE PRESENT INVENTION

The present invention provides methods of generating bone and/or cartilage in vivo, either ectopically or in situ, using growth factor biomolecules for activation and induction of stem cells that ultimately lead to the formation of bone and/or cartilage. Preferably, the growth factor biomolecules are delivered via biocompatible scaffolds.

In one embodiment, the methods of the invention include generating bone in vivo. The methods include administering a purified leukocyte cell-derived chemotaxin-1 (LECT-1; also called chondromodulin (ChM-1)) biomolecule, and a bone morphogenetic protein-2 (BMP-2) biomolecule to a subject in need thereof. The LECT-1 biomolecule can be a LECT-1 protein, or a nucleic acid molecule encoding a LECT-1 protein. The nucleic acid molecule can be either DNA gene or mRNA. The BMP-2 biomolecule can be a BMP-2 protein, or a nucleic acid molecule encoding a BMP-2 protein. The nucleic acid molecule can be either DNA gene or mRNA. The LECT-1 biomolecule and BMP-2 biomolecule can be administered sequentially or simultaneously.

In another embodiment, the methods of the invention include generating cartilage in vivo. The methods include administering a LECT-1 biomolecule to a subject in need thereof. Typically, a LECT-1 molecule is administered without a BMP-2 molecule when only cartilage (i.e., not bone) is to be generated.

In the methods of the invention, the LECT-1 and BMP-2 biomolecules treat damaged bones or regions lacking bone tissue by promoting, inducing and enhancing bone tissue regeneration. Without wanting to be held to a mechanism of action, it is believed that once the BMP-2 protein activates local adult mesenchymal cells (MSCs, repair cells of connective tissues including bones), the LECT-1 protein will induce progenitor cells differentiation into chondroblasts/chondrocytes to form the temporary and stabilizing endochondral cartilage/fibrocartilage callus. Cartilage tissues are naturally avascular; LECT-1 protein inhibits angiogenesis to direct cartilage formation. The regenerated cartilage cells will then undergo programmed cell death (apoptosis) but the tissue extracellular matrix (ECM) template is preserved and ossified by infiltrating bone cells.

Subjects who can benefit from the methods of the invention include mammals (e.g., humans and domestic mammals) who are in need of generating bone or cartilage. The bone to be generated can be any type of bone, for example, trabecular bone and cortical bone.

In some embodiments, the LECT-1 biomolecule, or the LECT-1 and BMP-2 biomolecules, is/are administered to a subject by injection or surgical implantation into an in vivo target site of the subject (e.g., by direct injection, implantation and/or placement into the affected tissue, in situ.) Examples of a target sites in situ for bone regeneration include a bone fracture, bone defect and bone gap. Examples of a target sites in situ for cartilage regeneration include cartilage damage/defect in the articular cartilage of joints, knee meniscus, costal cartilage, intervertebral disc of the spine and ear cartilage. In a preferred embodiment, the LECT-1 biomolecule, or the LECT-1 and BMP-2 biomolecules, is/are delivered via biocompatible scaffolds.

In other embodiments, instead of administering the biomolecule(s) directly into a target site, the biomolecule(s) are placed outside the cartilages and skeletal system to form cartilage and/or bone ectopically, thereby providing a source for autograft tissues. For example, a biocompatible scaffold of the present invention is placed into a subcutaneous layer/hypodermis/adipose tissue/subcutaneous white adipose tissue (sWAT)/under the skin, and/or within the muscle tissue for ectopic cartilage formation (i.e., chondrogenesis) or bone formation (i.e., osteogenesis), providing a source of autograft tissues. This autograft tissue, which is a cartilage and/or bone, is then harvested for use in a target cartilage and/or bone defect site.

In embodiments for bone regeneration, the LECT-1 protein can be administered at a range of about 5-125 μg/g target tissue weight; and the BMP-2 protein can be administered at a range of about 1-30 μg/g target tissue weight, when administered in the target region (site); at a BMP-2-to-LECT-1 molecule count ratio of about 100-2600:1. As would be known to a skilled artisan, for the administration of nucleic acids, dosing would depend on the specific features of the DNA/RNA vector used, including the specific promoters and other construct components. For example, for LECT-1 DNA/RNA, the dose can range from about 10 to 50 μg/g target tissue weight; and for BMP-2 DNA/RNA the dose can range from about 0.5 to 12.5 mg/g target tissue weight.

In embodiments for cartilage regeneration, the LECT-1 protein can be administered at a range of about 100-2500 μg/g target tissue weight; and the BMP-2 protein can be administered at a range of about 0-4 μg/g target tissue weight, when administered in the target region (site); at a BMP-2-to-LECT-1 molecule count ratio of about 0-16:1. As would be known to a skilled artisan, for the administration of nucleic acids, dosing would depend on the specific features of the DNA/RNA vector used, including the specific promoters and other construct components. For example, for LECT-1 DNA/RNA, the dose can range from about 200 to 1000 μg/g target tissue weight; and for BMP-2 DNA/RNA the dose can range from about 0 to 2 mg/g target tissue weight.

In one embodiment, the LECT-1 protein can be administered at a range of about 1-50 μg/kg body weight; and the BMP-2 protein can be administered at a range of about 100-600 μg/kg, when administered in the target region. In one embodiment, LECT-1 DNA/RNA can be administered in a dose range from about 0.5 to 20 μg/kg body weight; and BMP-2 DNA/RNA can be administered in a dose range from about 25 to 300 μg/kg body weight.

The development and growth of bone tissue is a complex biological process. Although not wanting to be bound by a mechanism of action, it is believed that in some embodiments of the invention, bone is generated via endochondral ossification, comprising forming intermediate cartilage via mesenchymal cells and progenitor cells (undifferentiated cells) differentiation into chondroblasts. The immediate cartilage is endochondral cartilage, articular cartilage, hyaline cartilage, elastic cartilage, fibrous cartilage and/or fibrocartilage. The immediate cartilage comprises cartilage-type cells. A cartilage-type cell includes chondroblast, fibrocartilage cell, and chondrocyte. The immediate cartilage then ossifies to bone. The bone comprises bone-type cells. A bone-type cell includes osteoblast and osteocyte, and tissue macrophage and osteoclast. Thus, in endochondral ossification, bone forms from a cartilage template. Most of the bones in the body, especially the long bones (e.g., femur, tibia, and humerus), develop through endochondral ossification. In this process, a cartilage tissue is converted and/or replaced with bone tissue. Chondrocytes (cartilage cells) within the cartilage tissue undergo hypertrophy (enlargement) and eventually die (i.e., apoptosis programmed cell death), leaving extracellular matrix (ECM) cavities that are invaded by blood vessels and cells (e.g., endothelial progenitor cells, pericytes, endothelial cells) and osteoblasts from differentiated mesenchymal stem cells. Osteoblasts then lay down bone matrix by calcifying the ECM and converting the cartilage into bone, also trapping the osteoblasts surrounded by the calcified matrix which terminally-differentiate into osteocytes.

In some embodiments of the invention, bone is generated via intramembranous ossification. In intramembranous ossification, bone forms directly within a membrane of connective tissue. This primarily occurs during the development of the flat bones of the skull, as well as some facial and clavicular bones. In intramembranous ossification, mesenchymal cells and progenitor cells (undifferentiated cells) differentiate directly into osteoblasts, which then produce bone matrix and ultimately form bone tissue.

The biomolecules of the present invention can be administered in vivo as a liquid in a carrier (e.g., in water and/or saline). In preferred embodiments, the biomolecules of the present invention are administered in vivo by biocompatible scaffolds. The scaffolds of the present invention can comprise, for example, a natural polymer, synthetic polymer, ceramic biomaterial, a porous metal biomaterial, and combinations thereof. Scaffolds are particularly preferred when producing autografts.

Examples of a natural polymer include a protein-based based polymer, a polysaccharide-based polymer, a proteoglycan and a proteoglycan complex. Examples of a protein-based based polymer include collagen, gelatin, fibrin, fibronectin, elastin, silk fibroin, hair protein extract and/or residual hair, laminin and polypeptides (e.g., polylysine and polyleucine). Hair protein extract and/or residual hair is keratin and/or keratin-associated proteins, with or without melanin pigments. Examples of polysaccharide-based polymers include non-sulfated glycosaminoglycans (GAGs) (e.g., hyaluronic acid), sulfated GAGs (e.g., chondroitin sulfate, dermatan sulfate, keratan sulfate, heparan sulfate, heparin) and other polysaccharides (e.g., chitosan, cellulose and its derivatives (e.g., cellulose acetate) dextran, alginic acid/alginate, extracellular matrix (ECM) extracts and decellularized tissue material). For instance, a commercial ECM is Matrigel® which is derived from a mouse tumor rich in laminin, collagen, and other ECM proteins. An example of a decellularized tissue is porcine small intestinal submucosa (SIS) obtained from decellularization process of a pig's SIS. Examples of proteoglycans include decorin, biglycan, testican, bikunin, fibromodulin, lumican, aggrecan, perlecan, betaglycan, agrin, neurocan, versican, and brevican. Examples of proteoglycan complexes include heparan sulfate proteoglycan, chondroitin sulfate proteoglycan, and keratan sulfate proteoglycan.

Examples of a synthetic polymer include polyethylene (PE), polypropylene (PP), polytetrafluoroethylene (PTFE), polyvinyl chloride (PVC), polydimethylsiloxane (PDMS)/silicone, polymethyl methacrylate (PMMA), polyhydroxyethyl methacrylate (PHEMA), polyethylene terephthalate (PET), nylon, polystyrene (PS), polyether ether ketone (PEEK), polyacrylic acid (PAA), polylactic acid (PLA), polyglycolic acid (PGA), poly(lactic-co-glycolic acid) (PLGA), poly(caprolactone) (PCL), polypropylene fumarate (PPF), polydioxanone (PDO), polyorthoester (POE), polyanhydride (PAH), polyurethane (PUR), polyamidoamine (PAA), polyvinyl alcohol (PVA), polyvinyl acetate (PVAc), poly(ethylene glycol) (PEG), modified PEG, polyacrylonitrile (PAN), polycyanoacrylate (PCA), poly(ethylene-vinyl acetate) (PEVA), polyacrylate, cyanoacrylate polymer, and combinations thereof.

Examples of a ceramic biomaterial include alumina, zirconia, metal oxides, carbon, carbides, glass−/−silica, silicon nitride, silicon carbide, porcelain (dental porcelain), bioglass−/− bioactive glass, calcium phosphates, hydroxyapatite (HA), carbonated HA, beta-tricalcium phosphate (TCP), calcium carbonate, glass ceramic composites (apatite-wollastonite deposited glass) and combinations thereof.

Examples of a porous metal biomaterial include titanium (Ti), titanium alloys (e.g., Ti-6Al-4V, Ti-6Al-7Nb, Ti-15Mo-5Zr-3Al, Ti-30Nb-5Ta-8Zr, Ti-35Zr-28Nb, and Ti-35Nb-2Ta-3Zr), nitinol (Ti—Ni)−/−shape memory alloy, tantalum (Ta), tantalum alloys, platinum (Pt), iron (Fe), zinc (Zn), magnesium (Mg), stainless steel, cobalt chromium alloys, and combinations thereof.

In the present invention, the LECT-1 and BMP-2 biomolecules are incorporated into a scaffold by covalent and/or non-covalent interactions. Covalent interactions create covalent bonds between the scaffold and the BMP-2 biomolecule and/or between the scaffold and the LECT-1 biomolecule, and/or within the scaffold itself. Covalent bonds make scaffolds less degradable, slow the rate of the activity of biomolecules, and allow for the control of the shape of scaffolds to better suit a specific target site. Examples of how a covalent interaction can be formed include subjecting and/or exposing the scaffold to a chemical reaction to increase the molecular weight by forming covalent or shared bonds (e.g., exposing a scaffold to glutaraldehyde), photo (light)-based reaction (e.g., 3D resin printing), thiol-ene reaction, disulfide oxidation reaction, crosslinking reaction using crosslinkers, and combinations thereof. Examples of how a non-covalent interaction can be formed include exposing the scaffold to physical mixing, dissolution, entanglement, absorption, adsorption, ionic or charged or electrostatic interaction, hydrophobic interaction, 3D filament printing, 3D resin printing, and combinations thereof. The formation of covalent or non-covalent interactions can be made during the formation of the scaffold and/or after the scaffold has already been formed. A skilled artisan would know techniques by which to form the covalent or non-covalent interactions.

The amino acid sequence of human LECT-1 protein is SEQ ID NO: 1, based on UniProt: >sp|O75829|CNMD_HUMAN Leukocyte cell-derived chemotaxin 1 OS═Homo sapiens OX=9606 GN═CNMD PE=1 SV=1

(SEQ ID NO: 1)
MTENSDKVPIALVGPDDVEFCSPPAYATLTVKPSSPARLLKVGAVVLISG
AVLLLFGAIGAFYFWKGSDSHIYNVHYTMSINGKLQDGSMEIDAGNNLET
FKMGSGAEEAIAVNDFQNGITGIRFAGGEKCYIKAQVKARIPEVGAVTKQ
SISSKLEGKIMPVKYEENSLIWVAVDQPVKDNSFLSSKVLELCGDLPIFW
LKPTYPKEIQRERREVVRKIVPTTTKRPHSGPRSNPGAGRLNNETRPSVQ
EDSQAFNPDNPYHQQEGESMTFDPRLDHEGICCIECRRSYTHCQKICEPL
GGYYPWPYNYQGCRSACRVIMPCSWWVARILGMV

A representative example of the nucleic acid sequence of human LECT is SEQ ID NO: 2, processed on https://www.bioinformatics.org/sms2/rev_trans.html and https://www.kazusa.or.jp/codon/cgi-bin/showcodon.cgi?species=9606&aa=1&style=GCG

(SEQ ID NO: 2)
ATGACCGAGAACAGCGACAAGGTGCCCATCGCCCTGGTGGGCCCCGACGA
CGTGGAGTTCTGCAGCCCCCCCGCCTACGCCACCCTGACCGTGAAGCCCA
GCAGCCCCGCCAGACTGCTGAAGGTGGGCGCCGTGGTGCTGATCAGCGGC
GCCGTGCTGCTGCTGTTCGGCGCCATCGGCGCCTTCTACTTCTGGAAGGG
CAGCGACAGCCACATCTACAACGTGCACTACACCATGAGCATCAACGGCA
AGCTGCAGGACGGCAGCATGGAGATCGACGCCGGCAACAACCTGGAGACC
TTCAAGATGGGCAGCGGCGCCGAGGAGGCCATCGCCGTGAACGACTTCCA
GAACGGCATCACCGGCATCAGATTCGCCGGCGGCGAGAAGTGCTACATCA
AGGCCCAGGTGAAGGCCAGAATCCCCGAGGTGGGCGCCGTGACCAAGCAG
AGCATCAGCAGCAAGCTGGAGGGCAAGATCATGCCCGTGAAGTACGAGGA
GAACAGCCTGATCTGGGTGGCCGTGGACCAGCCCGTGAAGGACAACAGCT
TCCTGAGCAGCAAGGTGCTGGAGCTGTGCGGCGACCTGCCCATCTTCTGG
CTGAAGCCCACCTACCCCAAGGAGATCCAGAGAGAGAGAAGAGAGGTGGT
GAGAAAGATCGTGCCCACCACCACCAAGAGACCCCACAGCGGCCCCAGAA
GCAACCCCGGCGCCGGCAGACTGAACAACGAGACCAGACCCAGCGTGCAG
GAGGACAGCCAGGCCTTCAACCCCGACAACCCCTACCACCAGCAGGAGGG
CGAGAGCATGACCTTCGACCCCAGACTGGACCACGAGGGCATCTGCTGCA
TCGAGTGCAGAAGAAGCTACACCCACTGCCAGAAGATCTGCGAGCCCCTG
GGCGGCTACTACCCCTGGCCCTACAACTACCAGGGCTGCAGAAGCGCCTG
CAGAGTGATCATGCCCTGCAGCTGGTGGGTGGCCAGAATCCTGGGCATGG
TG

The amino acid sequence of human BMP-2 protein is SEQ ID NO: 3, based on https://www.uniprot.org/uniprotkb/P12643/entry

(SEQ ID NO: 3)
MVAGTRCLLALLLPQVLLGGAAGLVPELGRRKFAAASSGRPSSQPSDEVL
SEFELRLLSMFGLKQRPTPSRDAVVPPYMLDLYRRHSGQPGSPAPDHRLE
RAASRANTVRSFHHEESLEELPETSGKTTRRFFFNLSSIPTEEFITSAEL
QVFREQMQDALGNNSSFHHRINIYEIIKPATANSKFPVTRLLDTRLVNQN
ASRWESFDVTPAVMRWTAQGHANHGFVVEVAHLEEKQGVSKRHVRISRSL
HQDEHSWSQIRPLLVTFGHDGKGHPLHKREKRQAKHKQRKRLKSSCKRHP
LYVDFSDVGWNDWIVAPPGYHAFYCHGECPFPLADHLNSTNHAIVQTLVN
SVNSKIPKACCVPTELSAISMLYLDENEKVVLKNYQDMVVEGCGCR

A representative example of the nucleic acid sequence of human BMP-2 protein is SEQ ID NO: 4, processed on https://www.bioinformatics.org/sms2/rev_trans.html and https://www.kazusa.or.jp/codon/cgi-bin/showcodon.cgi?species=9606&aa=1&style=GCG

(SEQ ID NO: 4)
ATGGTGGCCGGCACCAGATGCCTGCTGGCCCTGCTGCTGCCCCAGGTGCT
GCTGGGCGGCGCCGCCGGCCTGGTGCCCGAGCTGGGCAGAAGAAAGTTCG
CCGCCGCCAGCAGCGGCAGACCCAGCAGCCAGCCCAGCGACGAGGTGCTG
AGCGAGTTCGAGCTGAGACTGCTGAGCATGTTCGGCCTGAAGCAGAGACC
CACCCCCAGCAGAGACGCCGTGGTGCCCCCCTACATGCTGGACCTGTACA
GAAGACACAGCGGCCAGCCCGGCAGCCCCGCCCCCGACCACAGACTGGAG
AGAGCCGCCAGCAGAGCCAACACCGTGAGAAGCTTCCACCACGAGGAGAG
CCTGGAGGAGCTGCCCGAGACCAGCGGCAAGACCACCAGAAGATTCTTCT
TCAACCTGAGCAGCATCCCCACCGAGGAGTTCATCACCAGCGCCGAGCTG
CAGGTGTTCAGAGAGCAGATGCAGGACGCCCTGGGCAACAACAGCAGCTT
CCACCACAGAATCAACATCTACGAGATCATCAAGCCCGCCACCGCCAACA
GCAAGTTCCCCGTGACCAGACTGCTGGACACCAGACTGGTGAACCAGAAC
GCCAGCAGATGGGAGAGCTTCGACGTGACCCCCGCCGTGATGAGATGGAC
CGCCCAGGGCCACGCCAACCACGGCTTCGTGGTGGAGGTGGCCCACCTGG
AGGAGAAGCAGGGCGTGAGCAAGAGACACGTGAGAATCAGCAGAAGCCTG
CACCAGGACGAGCACAGCTGGAGCCAGATCAGACCCCTGCTGGTGACCTT
CGGCCACGACGGCAAGGGCCACCCCCTGCACAAGAGAGAGAAGAGACAGG
CCAAGCACAAGCAGAGAAAGAGACTGAAGAGCAGCTGCAAGAGACACCCC
CTGTACGTGGACTTCAGCGACGTGGGCTGGAACGACTGGATCGTGGCCCC
CCCCGGCTACCACGCCTTCTACTGCCACGGCGAGTGCCCCTTCCCCCTGG
CCGACCACCTGAACAGCACCAACCACGCCATCGTGCAGACCCTGGTGAAC
AGCGTGAACAGCAAGATCCCCAAGGCCTGCTGCGTGCCCACCGAGCTGAG
CGCCATCAGCATGCTGTACCTGGACGAGAACGAGAAGGTGGTGCTGAAGA
ACTACCAGGACATGGTGGTGGAGGGCTGCGGCTGCAGA

The degeneracy of the genetic code further widens the scope of the embodiments as it enables major variations in the nucleotide sequence of a DNA molecule while maintaining the amino acid sequence of the encoded protein. The “degeneracy” means that a different three-letter codon can be used to specify a particular amino acid. For example, alanine can be encoded in the cDNA (complementary DNA or DNA synthesized form of the mature mRNA) by the nucleotide codon triplet GCT; however, because of the degeneracy of the genetic code, three other nucleotide codon triplets—GCT, GCC and GCA—also code for alanine. Thus, the nucleotide sequence of LECT protein could be changed at this position to any of these three codons without affecting the amino acid composition of the encoded protein or the characteristics of the protein. The genetic code and variations in nucleotide codons for particular amino acids are well known to the skilled artisan. For example, it is well known in the art that the following codons can be used interchangeably to code for each specific amino acid:

| | |
|---|---|
| Phenylalanine (Phe | UUU or UUC |
| or F) | |
| Leucine (Leu or L) | UUA or UUG or CUU or CUC or CUA or CUG |
| Isoleucine (Ile or I) | AUU or AUC or AUA |
| Methionine (Met | AUG |
| or M) | |
| Valine (Val or V) | GUU or GUC of GUA or GUG |
| Serine (Ser or S) | UCU or UCC or UCA or UCG or AGU or AGC |
| Proline (Pro or P) | CCU or CCC or CCA or CCG |
| Threonine (Thr or T) | ACU or ACC or ACA or ACG |
| Alanine (Ala or A) | GCU or GCG or GCA or GCG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp | GAU or GAC |
| or D) | |
| Glutamic Acid (Glu | GAA or GAG |
| or E) | |
| Cysteine (Cys or C) | UGU or UGC |
| Arginine (Arg or R) | CGU or CGC or CGA or CGG or AGA or AGG |
| Glycine (Gly or G) | GGU or GGC or GGA or GGG |
| Tryptophan (Trp or W) | UGG |
| Termination codon | UAA (ochre) or UAG (amber) or UGA (opal) |

Also, it should be understood that the codons specified above are for DNA sequences. The corresponding codons for RNA have a U substituted for T.

As various changes may be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

1. A method of generating bone in vivo, the method comprising:
administering leukocyte cell-derived chemotaxin-1 (LECT-1) biomolecule and bone morphogenetic protein-2 (BMP-2) biomolecule into an in vivo target site,
whereby bone is generated in the in vivo target site.

2. The method of claim 2 wherein LECT-1 and BMP-2 molecules are administered by placing the biomolecules into a biocompatible scaffold, wherein the scaffold comprises a natural polymer, synthetic polymer, ceramic biomaterial, porous metal biomaterial and combinations thereof.

3. The method of claim 2 wherein the natural polymer is a protein-based based polymer, polysaccharide-based polymer, and combinations thereof.

4. The method of claim 3 wherein the protein-based based polymer is: collagen, gelatin, fibrin, fibronectin, elastin, silk fibroin, hair protein extract, residual hair, laminin, polypeptides and combinations thereof.

5. The method of claim 3 wherein the polysaccharide-based polymer is a chitosan, cellulose, cellulose acetate, dextran, alginic acid/alginate, non-sulfated glycosaminoglycan (GAG), sulfated GAG, proteoglycans, proteoglycan complex, extracellular matrix (ECM) extracts and/or decellularized tissue ECM material, and combinations thereof.

6. The method of claim 5 wherein the non-sulfated GAG is hyaluronic acid, wherein the sulfated GAG is chondroitin sulfate, dermatan sulfate, keratan sulfate, heparan sulfate, heparin and combinations thereof, wherein the proteoglycan is decorin, biglycan, testican, bikunin, fibromodulin, lumican, aggrecan, perlecan, betaglycan, agrin, neurocan, versican, brevican and combinations thereof, and wherein the proteoglycan complex is heparan sulfate proteoglycan, chondroitin sulfate proteoglycan, keratan sulfate proteoglycan, and combinations thereof.

7. The method of claim 2 wherein the synthetic polymer is polyethylene (PE), polypropylene (PP), polytetrafluoroethylene (PTFE), polyvinyl chloride (PVC), polydimethylsiloxane (PDMS), polymethyl methacrylate (PMMA), polyhydroxyethyl methacrylate (PHEMA), polyethylene terephthalate (PET), nylon, polystyrene (PS), polyether ether ketone (PEEK), polyacrylic acid (PAA), polylactic acid (PLA), polyglycolic acid (PGA), poly(lactic-co-glycolic acid) (PLGA), poly(caprolactone) (PCL), polypropylene fumarate (PPF), polydioxanone (PDO), polyorthoester (POE), polyanhydride (PAH), polyurethane (PUR), polyamidoamine (PAA), polyvinyl alcohol (PVA), polyvinyl acetate (PVAc), poly(ethylene glycol) (PEG), modified PEG, polyacrylonitrile (PAN), polycyanoacrylate (PCA), poly(ethylene-vinyl acetate) (PEVA), polyacrylate, cyanoacrylate polymer, and combinations thereof.

8. The method of claim 2 wherein the ceramic biomaterial is alumina, zirconia, metal oxides, carbon, carbides, glass−/−silica, silicon nitride, silicon carbide, porcelain, bioglass−/− bioactive glass, calcium phosphates, hydroxyapatite (HA), carbonated HA, beta-tricalcium phosphate (TCP), calcium carbonate, glass ceramic composites and combinations thereof.

9. The method of claim 2 wherein the porous metal biomaterial is titanium, titanium alloys, nitinol (Ti—Ni)−/−shape memory alloy, tantalum, tantalum alloys, platinum, iron, zinc, magnesium, stainless steel, cobalt chromium alloys, and combinations thereof.

10. The method of claim 1 wherein the LECT-1 and BMP-2 biomolecules are incorporated into the scaffold by covalent and/or non-covalent interactions.

11. The method of claim 10 wherein the covalent interaction creating covalent bonds between the scaffold and BMP-2 and/or between the scaffold and LECT-1 and/or within the scaffold itself comprises exposing the scaffold to a photo (light)-based reaction, thiol-ene reaction, disulfide oxidation reaction, crosslinking reaction using crosslinkers and combinations thereof.

12. The method of claim 10 wherein the non-covalent interaction comprises exposing the scaffold to physical mixing, dissolution, entanglement, absorption, adsorption, ionic or charged or electrostatic interaction, hydrophobic interaction, 3D filament printing, 3D resin printing and combinations thereof.

13. The method of claim 1,
wherein the target site is a bone fracture, bone defect, bone gap; or
wherein the target site is a skin layer, under the skin or within muscle tissue to provide a source for autograft bone tissue, wherein the skin layer is a subcutaneous layer, hypodermis, adipose tissue, subcutaneous or white adipose tissue (sWAT).

14. The method of claim 1 wherein the LECT-1 biomolecule is a LECT-1 protein and/or a nucleic acid molecule encoding a LECT-1 protein.

15. The method of claim 1 wherein the BMP-2 biomolecule is a BMP-2 protein and/or a nucleic acid molecule encoding a BMP-2 protein.

16. A method of generating cartilage in vivo, the method comprising:
providing a biocompatible scaffold containing leukocyte cell-derived chemotaxin-1 (LECT-1) biomolecule and
placing the biocompatible scaffold into an in vivo target site,
wherein cartilage is generated within the target site.

17. The method of claim 16 wherein the biocompatible scaffold further contains bone morphogenetic protein-2 (BMP-2) biomolecule.

18. The method of claim 16 wherein the cartilage is articular cartilage, endochondral cartilage, hyaline cartilage, elastic cartilage, fibrous cartilage/fibrocartilage and combinations thereof.

19. The method of claim 16,
wherein the target site is a cartilage defect, cartilage gap for cartilage regeneration,
wherein the target site is a skin layer, under the skin or within muscle tissue to provide a source for autograft cartilage tissue, wherein the skin layer is a subcutaneous layer, hypodermis, adipose tissue, subcutaneous or white adipose tissue (sWAT).

20. A biocompatible scaffold containing leukocyte cell-derived chemotaxin-1 (LECT-1) biomolecule and bone morphogenetic protein-2 (BMP-2) biomolecule, for generating bone and/or cartilage in vivo.
